(19) United States
(12) Patent Application Publication
(10) Pub. No.: US 2007/0087438 A1
(43) Pub. Date: Apr. 19, 2007

(54) Methods for conditioning plant somatic embryos

(76) Inventors: James A. Grob, Bonnie Lake, WA (US); Patrick M. Brownell, Tacoma, WA (US)

Correspondence Address: WEYERHAEUSER COMPANY;INTELLECTUAL PROPERTY DEPT., CH 1J27, P.O. BOX 9777, FEDERAL WAY, WA, 98063, US

(21) Appl. No.: 11/534,576

(22) Filed: Sep. 22, 2006

Related U.S. Application Data
(60) Provisional application No. 60/727,373, filed on Oct. 17, 2005.

(51) Int. Cl. C12N 15/82; C12N 5/04

(52) U.S. Cl. 435/468; 435/419

(57) ABSTRACT

The present invention provides methods for conditioning plant somatic embryos. The methods include the step of exposing the somatic embryos to a gas stream having a selected moisture content for a period of time sufficient to change the moisture content of the somatic embryo to a desired moisture content, wherein the gas stream is produced using an ionomeric membrane.

### CROSS REFERENCE TO RELATED APPLICATION

The present application claims the benefit of U.S. Provisional Application No. 60/727,373, filed Oct. 17, 2005.

### FIELD OF THE INVENTION

The present invention relates to methods for conditioning plant tissue, in particular plant somatic embryos.

### BACKGROUND OF THE INVENTION

The demand for coniferous trees, such as pines and firs, to make wood products continues to increase. One proposed solution to this problem is to identify individual trees that possess desirable characteristics, such as a rapid rate of growth, and produce numerous, genetically identical, clones of the superior trees by somatic cloning.

Somatic cloning is the process of creating genetically identical trees from tree tissue other than the male and female gametes. In one approach to somatic cloning, plant tissue is cultured in an initiation medium which includes hormones, such as auxins and/or cytokinins, that initiate formation of embryogenic cells that are capable of developing into somatic embryos. The embryogenic cells are then further cultured in a maintenance medium that promotes multiplication of the embryogenic cells. The multiplied embryogenic cells are then cultured in a development medium that promotes development of cotyledonary somatic embryos which can, for example, be placed within artificial seeds and sown in the soil where they germinate to produce conifer seedlings. The seedlings can be transplanted to a growth site for subsequent growth and eventual harvesting to yield lumber, or wood-derived products.

Loblolly pine (Pinus taeda) is an important conifer species that can be reproduced by somatic cloning. Loblolly pine somatic embryos appear to be physically mature at the end of the development stage of the somatic cloning process, but subsequent periods of incubation in the cold (at a temperature of about 4° C.) and conditioning are required to promote physiological maturation of the embryos. Without cold treatment and conditioning the germination efficiency of a population of somatic embryos is low.

One method for conditioning conifer somatic embryos is to incubate the embryos in a closed, gas-tight, container in the presence of water or a salt solution. The embryos are not in direct physical contact with the water, or salt solution, but are exposed to the humidified atmosphere created by the water, or salt solution, for a period of time sufficient to dehydrate the embryos to a desired moisture content. Although this dehydration method is effective, it is nonetheless difficult to control the amount of dehydration of the embryos, and the rate of dehydration is determined by the time taken for the surrounding atmosphere and embryos to come into equilibrium with the water or salt solution.

There is a continuing need for methods for conditioning plant somatic embryos, such as conifer somatic embryos, to promote maturation and germination thereof.

### SUMMARY OF THE INVENTION

The present inventors have discovered that ionomeric membranes can be used to adjustably control the moisture content of gas that is used to condition plant somatic embryos in order to promote physiological maturation of the embryos. Thus, in one aspect, the present invention provides methods for conditioning a plant somatic embryo. The methods of this aspect of the invention each include the step of exposing the somatic embryo to a gas stream having a selected moisture content for a period of time sufficient to change the moisture content of the somatic embryo to a desired moisture content, wherein: (a) the gas stream is produced by using an ionomeric membrane comprising a membrane body defining a first surface and a second surface; (b) the first surface is contacted with an aqueous liquid, and the second surface is contacted with moving gas; (c) the membrane body permits water to move from the first surface to the second surface; and (d) the first surface of the ionomeric membrane is contacted with the aqueous liquid for a period of time sufficient to permit enough water to cross the membrane to change the moisture content of the moving gas to produce the gas stream having the selected moisture content.

The methods of the present invention are useful, for example, for conditioning conifer somatic embryos, such as loblolly pine and Douglas-fir somatic embryos, to promote physiological maturation of the somatic embryos, and thereby improve the germination rate of the somatic embryos.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows an example of a system for conditioning plant somatic embryos.

FIG. 2 shows a longitudinal cross sectional view of a metal tube, containing a Nafion® tube, that is used in the system shown in FIG. 1 to alter the moisture content of gas that is used to dry plant somatic embryos.

FIG. 3 shows a graph of percent relative humidity of gas versus time (minutes). The moisture content of the gas was increased by increasing the temperature of water flowing over a semi-permeable Nafion® membrane, as described more fully herein. The following abbreviations are used: RH means relative humidity; temp means temperature.

FIG. 4 shows a graph of percent relative humidity of gas versus time (minutes). The moisture content of the gas was decreased by decreasing the temperature of water flowing over a semi-permeable Nafion® membrane, as described more fully herein. The following abbreviations are used: RH means relative humidity; temp means temperature.

### DETAILED DESCRIPTION OF THE PREFERRED EMBODIMENT

The present invention provides methods for conditioning a plant somatic embryo. The methods each include the step of exposing the somatic embryo to a gas stream having a selected moisture content for a period of time sufficient to change the moisture content of the somatic embryo to a desired moisture content, wherein: (a) the gas stream is produced by using an ionomeric membrane comprising a membrane body defining a first surface and a second surface; (b) the first surface is contacted with an aqueous liquid, and the second surface is contacted with moving gas; (c) the membrane body permits water to move from the first surface to the second surface; and (d) the first surface of the ionomeric membrane is contacted with the aqueous liquid for a period of time sufficient to permit enough water to cross the membrane to change the moisture content of the moving gas to produce the gas stream having the selected moisture content.

The term “condition” or “conditioning,” when used in connection with a plant somatic embryo, means promoting physiological maturation of the somatic embryo by changing the moisture content of the somatic embryo (e.g., by increasing or decreasing the moisture content of the somatic embryo).

For ease of description, the present invention is described with reference to conditioning a single somatic embryo. It will be understood, however, that typically in the practice of the present invention numerous somatic embryos (e.g., tens, or hundreds, or thousands) are dried together.

An ionomeric membrane is a membrane made from an ionomer. Ionomers are copolymers that contain non-ionic repeat units and a small amount (typically less than 15% by weight of the ionomer) of ion-containing repeat units. Non-covalent bonds form between the ion-containing repeat units on different copolymer chains, while covalent bonds occur between non-ionic portions of the copolymer. The ionomers useful in the practice of the present invention are semi-permeable membranes that permit water to pass through the membrane.

Nafion® is an example of an ionomer that is useful in the practice of the present invention. Nafion® is a copolymer of tetrafluoroethylene (Teflon®) and perfluoro-3,6-dioxa-4-methyl-7-octene-sulfonic acid. Nafion® is highly resistant to chemical attack, moreover sulfonic acid has a very high water-of-hydration, absorbing 13 molecules of water for every sulfonic acid group in the polymer, and so Nafion® absorbs 22% by weight of water. Nafion® moves water by absorption as water-of-hydration. This absorption occurs as a first order kinetic reaction, so equilibrium is reached very quickly (typically within milliseconds) and is proportional to temperature. Nafion® membrane is commercially available, for example from DuPont, 1007 Market Street, Wilmington, Del. 19898, U.S.A.

In the practice of the present invention, a plant somatic embryo is exposed to a gas stream having a selected moisture content for a period of time sufficient to dry the somatic embryo to a desired moisture content. For example, plant somatic embryos can be dried to a moisture content in the range of from 10% to 80%.

The amount of time that the somatic embryo is exposed to the gas stream in order to dry the somatic embryo to a desired moisture content depends on such factors as the starting moisture content of the embryo, the desired moisture content end point, the relative humidity of the gas stream, and the convective flow rate of the gas stream.

By way of non-limiting example, gymnosperm somatic embryos can be conditioned by exposing the embryos to moving gas that flows at a rate in the range of from less than 1 liter/minute to 30 liters/minute, having a relative humidity in the range of from 30% to 100%, for a period of from 1 hour to 12 weeks. The temperature of the gas stream can be, for example, in the range of from 15° C. to 30° C.

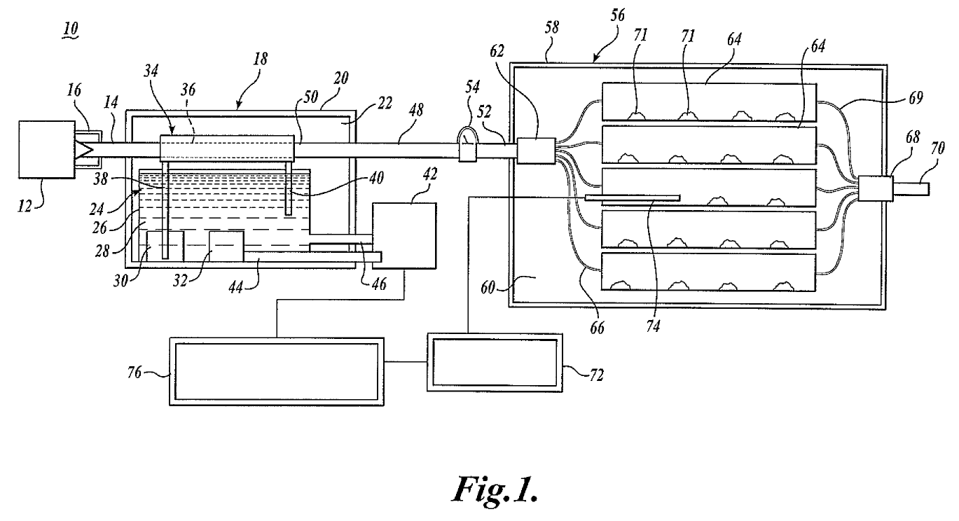

FIG. 1 shows an example of a system 10 for conditioning plant somatic embryos. System 10 includes a gas pump 12 that pumps gas through a first tube portion 14. Gas flow from pump 12 is regulated by a gas flow regulator 16. System 10 also includes a first insulated box 18 that includes a box body 20 that defines a box interior space 22. A water container 24 is located within box interior space 22. Water container 24 includes a water container body 26 that defines a water container interior space 28 that is adapted to contain water. A first water pump 30 and a second water pump 32 are located within water container interior space 28.

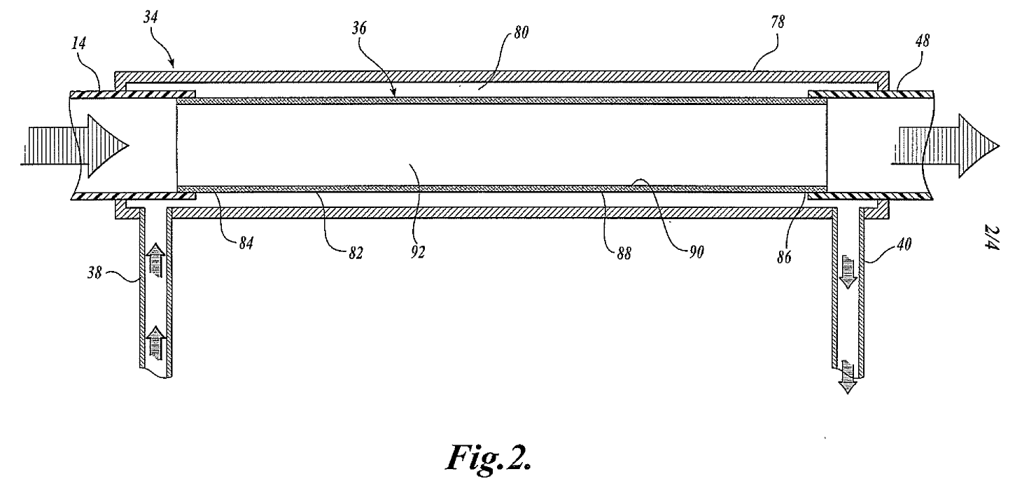

A metal tube 34 is located within box interior space 22 but outside water container 24. As shown more clearly in FIG. 2, a Nafion® tube 36 is disposed within metal tube 34. Metal tube 34 is connected to first water pump 30 by first water pump tube 38. An outflow tube 40 connects metal tube 34 with water container interior space 28.

System 10 includes a water temperature regulator 42 located outside first insulated box 18. A second pump tube 44 connects second water pump 32 to water temperature regulator 42. A temperature regulator tube 46 connects water temperature regulator 42 to water container interior space 28.

A second tube portion 48 includes a first end 50 and a second end 52. A gas flow meter 54 is located on second tube portion 48. Second tube portion 48 connects metallic tube 34 to a second insulated box 56 that includes a second insulated box body 58 that defines a second insulated box interior space 60. A first gas filter 62 is mounted on second end 52 of second tube portion 48 and is located within second insulated box interior space 60. A multiplicity of sterile boxes 64 are also located within second insulated box interior space 60. Each sterile box 64 is connected to first gas filter 62 by a first conduit 66, such as autoclavable silicon tubing. System 10 also includes a second gas filter 68 that penetrates second insulated box body 58. A multiplicity of second conduits 69 connect sterile boxes 64 to second gas filter 68. An exhaust tube 70 is connected to second gas filter 68. Plant somatic embryos 71 are shown in sterile boxes 64.

System 10 also includes a relative humidity reader 72 that is electrically connected to a relative humidity probe 74 disposed within sterile box 64. An electronic feedback controller 76 is electrically connected to water temperature regulator 42 and to relative humidity reader 72.

FIG. 2 shows a longitudinal cross-sectional view of metal tube 34. Metal tube 34 includes a metal tube body 78 that defines a metal tube interior space 80. Nafion® tube 36 is disposed within metal tube interior space 80. Nafion® tube 36 includes a Nafion® tube body 82 having a first end 84 and a second end 86. Nafion® tube body first end 84 is connected to first tube portion 14, and Nafion® tube body second end 86 is connected to second tube portion 48. Nafion® tube body 82 defines a first (outer) surface 88 and a second (inner) surface 90. Nafion® tube body 82 also defines a Nafion® tube body internal space 92.

In operation, gas pump 12 pumps gas through first tube portion 14 into Nafion® tube body internal space 92. Gas flow into Nafion® tube body internal space 92 is regulated by gas flow regulator 16. First water pump 30 pumps water through first water pump tube 38 into metal tube interior space 80. The pumped water flows around Nafion® tube 36 and contacts Nafion® tube body first surface 88. Some water passes from Nafion® tube body first surface 88, through Nafion® tube body 82, and through Nafion® tube body second surface 90 into the gas passing through Nafion® tube body internal space 92, thereby humidifying the gas. Water leaves metal tube interior space 80 through outflow tube 40 which directs the outgoing water back into water container interior space 28.

The water in water container interior space 28 is maintained at a desired temperature, or maintained within a desired temperature range, by water temperature regulator 42. Second water pump 32 pumps water from water container interior space 28, through second pump tube 44, into water temperature regulator 42, and out of water temperature regulator 42, through water temperature regulator tube 46, back into water container interior space 28.

Moistened gas leaves Nafion® tube body internal space 92 through second tube portion 48 and passes through first gas filter 62 which removes particulate matter, such as bacteria, from the gas. Gas flow meter 54 monitors the amount of gas passing through second tube portion 48. Gas leaves first gas filter 62 and enters multiplicity of first conduits 66 that direct the gas into multiplicity of sterile boxes 64. Each sterile box 64 contains plant somatic embryos 71. Gas passes over plant somatic embryos 71 and dries them to a desired moisture content. The gas leaves sterile boxes 64 through multiplicity of second conduits 69 and passes through second gas filter 68. Second gas filter 68 prevents accidental reversal of gas flow carrying microbes into second insulated box interior space 60.

The humidity of the gas passing through sterile boxes 64 is measured using relative humidity probe 74 that is electrically connected to relative humidity reader 72. The humidity of the gas passing through sterile boxes 64 is controlled by electronic feedback controller 76 which receives information regarding the humidity of the gas in sterile boxes 64 from relative humidity reader 72. When the humidity within sterile boxes 64 drops below a desired level, then electronic feedback controller 76 electrically stimulates water temperature regulator 42 to increase the temperature of water in water container 24. The increased water temperature promotes movement of water across Nafion® tube body 82, and thereby increases the moisture content of the gas flowing through Nafion® tube body internal space 92 and then into sterile boxes 64. Conversely, when the humidity within sterile boxes 64 rises above a desired level, then electronic feedback controller 76 electrically stimulates water temperature regulator 42 to decrease the temperature of water in water container 24. The decreased water temperature reduces the rate of movement of water across Nafion® tube body 82, and thereby decreases the moisture content of the gas flowing through Nafion® tube body internal space 92 and then into sterile boxes 64.

### EXAMPLES

### Example 1

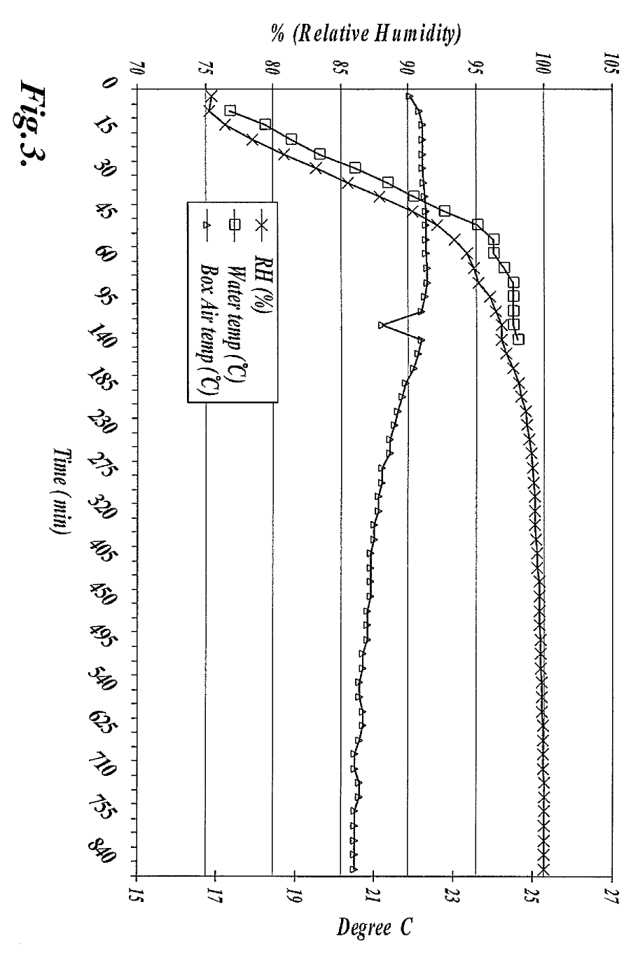

This Example describes the system used to obtain the results shown in FIG. 3 which shows that an increase in the temperature of water flowing around a Nafion® tube causes a corresponding increase in the relative humidity of gas flowing out of the Nafion® tube.

The following system was used in this and subsequent Examples, unless stated otherwise. A controlled water temperature bath system was built using a Teclima Micro chiller/heater/controller (Fritz Industries, 500 Sam Houston Rd., Mesquite, Tex. 75149). Temperature stability was achieved by using a large thermal mass of water (4 gallons) and by placing the water in a 5 gallon water can that sat in a well insulated cooler. Water was moved through the Teclima heater/chiller using a standard aquarium pump (Aquarium Systems Mini-Jet 606) that provided a flow rate of about 60 gallons per hour.

Water was pumped through the Nafion® system using a separate Mini-Jet pump which provided a fast drip of water through a ⅛ inch tube that fed the Nafion® tube. Gas flow through the Nafion® tube was provided by a flow-rate adjustable Rena Gas 400 aquarium gas pump. This pump was calibrated using a Fischer and Porter Flowmeter kit. Flow rate was adjustable from 150 cc/min to 1500 cc/min (wherein “cc” is the abbreviation for cubic centimeters).

The Nafion® tube was a single 0.05 inch diameter, 12 inch Nafion tube humidifier in a stainless steel tube (single MH-050-12-S tube with ⅛ inch ID inlets and outlets). The conditioning container, that housed the somatic embryos, was a half Cambro™ box (Cambro Manufacturing, Huntington Beach, Calif.) with gasketed lid that was modified to accept a Vaisala relative humidity probe. The box was kept closed with four clips. Water testing the seals demonstrated the box was not water tight under the slight positive pressure of the gas flow.

Water and box gas temperature, and relative humidity, were manually quantified during periods of system change by reading the digital outputs on the Teclima and Vaisala screens. During long periods of static conditions, box gas and relative humidity was recorded by the Vaisala probe at 15 minute intervals.

### Example 2

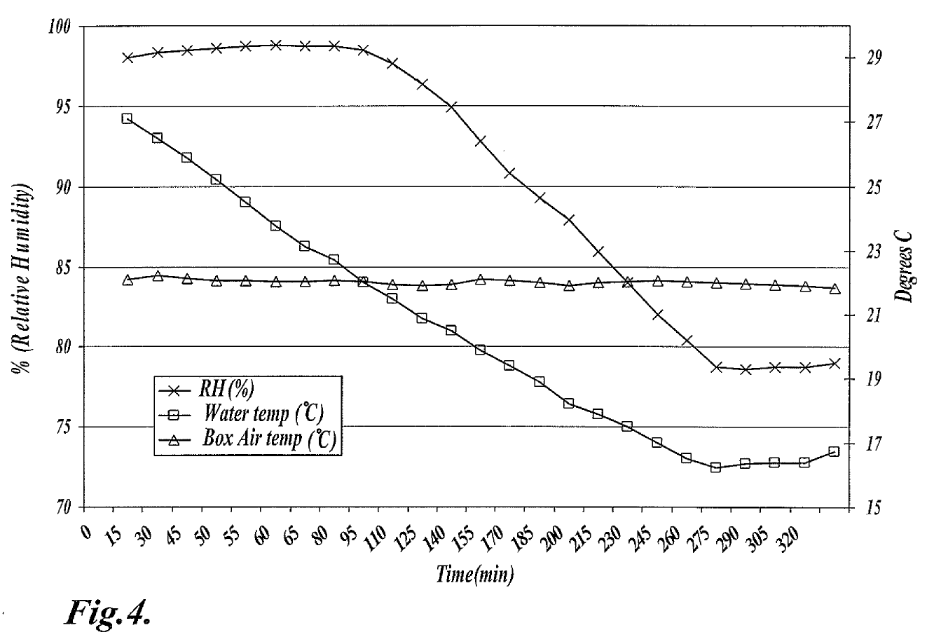

In this example, the system described in Example 1 was used to generate the data shown in FIG. 4, which shows that a decrease in the temperature of water flowing around the Nafion® tube causes a corresponding decrease in the relative humidity of gas flowing out of the Nafion® tube and into the half Cambro box. In this test gas flow was 1 liter/min, water bath was (˜4 gallons), and the water temperature set point was changed from 29° C. to 20° C. The relative humidity of the gas was not affected until the temperature of the water flowing around the Nafion® tube was reduced to below the temperature of the gas in the half Cambro box.

### Example 3

This Example demonstrates how the combination of the relative humidity of a gas stream and the flow rate of the humidified gas can modulate plant somatic embryo moisture content within a 24 hour period.

The experiments reported in this example used a modified version of the system described in Example 1. A multiplexed Nafion tube PH-30T-12PS (Perma Pure Inc, Toms River, N.J.) was used which greatly increased the surface area for water exchange under high gas flow rates. The aquarium pump was replaced with building forced air that had a moisture content of about 10%, or less, and provided much greater flow rate. The embryo conditioning box was contained in a Styrofoam box to reduce the effect of room temperature changes on chamber temperature. An electronic controller was added to control water bath temperature so that a pre-programmed temperature differential would exist between water bath and conditioning box. In these experiments, sterile filters were not used because sterility was not required during the course of these experiments.

The controller consisted of a single mode proportion control used to control the water bath temperature to ±0.1° C. Water was circulated with a pump through the Nafion tube and then to a heat exchanger that heated or cooled the water. This water control loop had a high gain and had a cycle time of about 5 seconds. The high gain keeps the response time fast. The set point for the controller was determined by an offset the operator entered between the temperature of the test chamber and that in the water bath.

The following set of experiments varied both the temperature differential between water bath and conditioning box in order to create a varied relative humidity gas stream and convective water loss/gain.

Treatments and results are summarized in TABLE 1.

TABLE 1

| | | | | Average temperature | | |
|---|---|---|---|---|---|---|
| | | | | differential (° C.) | | |
| | Test Duration | Average gas | Average Flow | between chamber and | Starting Moisture | Ending Moisture |
| Run Number | (hr) | RH (%) | Rate (l/min) | water bath | content (%) | content (%) |
| Run 1 | 24 hours | 100.4 | 1.0 | −0.6 | 75.8 | 73.4 |
| Run 2 | 24 hours | 98.2 | 1.1 | 1.1 | 76.4 | 69.3 |
| Run 3 | 24 hours | 99.7 | 9.0 | 0.2 | 75.2 | 69.6 |
| Run 4 | 24 hours | 94.3 | 10.6 | −0.8 | 75.1 | 26.8 |
| Run 5 | 24 hours | 96.9 | 10.6 | −0.3 | 74.3 | 42.1 |
| Run 6 | 24 hours | 99.3 | 10.0 | 0.3 | 42.1 | 57.2 |

The abbreviations used in Table 1 are: hr (hour); RH (relative humidity); l/min (liters per minute). Somatic embryos of loblolly pine were grown in liquid culture then plated for a development period of 12 weeks, followed by 4 weeks on a stratification medium at 4° C. At the end of this sequence, embryos were placed onto a nylon membrane that was stretched across a metal frame. Frames were either temporarily stored over water in a Cambro box, or placed in a Cambro box attached to the Nafion system described herein.

Before each run, a sample of 60 embryos was taken for moisture content analysis to determine a starting point for the experiment. Briefly this consisted of 3 replications of approximately 20 embryos that were rapidly placed into a tared ground glass vial to determine a fresh weight, then drying embryos (caps off) at 60° C. in an oven for 24-48 hours, or until dry weight was stable. At time of dry weight determination, vials were removed and capped and allowed to return to room temperature before weighing. A second moisture content sampling for both wet and dry weight was then taken at the end of the conditioning period. Moisture content was determined by the following equation:

$$\frac{( Wet weight - dry weight )}{Wet weight} \times 100$$

These experiments demonstrated that the method is capable of reducing, increasing, and/or maintaining the moisture content of the embryo. For example, in run 1 moisture content was minimally affected by conditioning for 24 hours, changing only 2%, while in runs 2-6 ending moisture content varied from 27-69%. Comparison of run 2 and 3 demonstrates that a similar moisture content reduction can occur by either an alteration in relative humidity or flow rate. Runs 4 and 5 show that small changes in relative humidity at high flow rates can have substantial effects on moisture content of embryos. Lastly, comparison of runs 5 and 6 demonstrates that, once embryos are dried to low moisture content, such as in run 5, increasing relative humidity of the gas stream causes a 15% increase in embryo moisture content. Thus, the system can be used as a means of controlled embryo hydration. Taken together these runs are an excellent example of the general ability and flexibility of the system in controlling embryo moisture content.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for conditioning a plant somatic embryo, the method comprising the step of exposing the somatic embryo to a gas stream having a selected moisture content for a period of time sufficient to change the moisture content of the somatic embryo to a desired moisture content, wherein:
(a) the gas stream is produced by using an ionomeric membrane comprising a membrane body defining a first surface and a second surface;
(b) the first surface is contacted with an aqueous liquid, and the second surface is contacted with moving gas;
(c) the membrane body permits water to move from the first surface to the second surface; and
(d) the first surface of the ionomeric membrane is contacted with the aqueous liquid for a period of time sufficient to permit enough water to cross the membrane to change the moisture content of the moving gas to produce the gas stream having the selected moisture content.

2. A method of claim 1 wherein the somatic embryo is a gymnosperm somatic embryo.

3. A method of claim 1 wherein the somatic embryo is a conifer somatic embryo.

4. A method of claim 1 wherein the somatic embryo is a loblolly pine somatic embryo.

5. A method of claim 1 wherein the somatic embryo is a Douglas-fir somatic embryo.

6. A method of claim 1 wherein the selected moisture content of the gas stream is a relative humidity from 30% to 100%.

7. A method of claim 1 wherein the somatic embryo is exposed to the gas stream for a period of time of from 1 hour to 12 weeks.

8. A method of claim 1 wherein the gas stream has a temperature in the range of from 15° C. to 30° C.

9. A method of claim 1 wherein the ionomeric membrane is a perfluorinated sulfonic acid polymer membrane.

10. A method of claim 1 wherein the moisture content of the somatic embryo is increased.

11. A method of claim 1 wherein the moisture content of the somatic embryo is decreased.

12. A method of claim 1 wherein the gas stream consists essentially of air.

13. A method of claim 1 wherein the aqueous liquid consists essentially of water.

14. A method of claim 1 wherein the ionomeric membrane is in the form of a tube.
